SEQUENCE LISTING (17 sequences)

SEQ ID NO: 1 - protein, 223 aa, Artificial Sequence
  Synthetic Peptide
MACLGFQRHKAQLNLATRTWPCTLLFFLLFIPVFCKAMHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIDPEPCPDSDFLLWILAAVSSGLFFYSFLLTAVSLSKMLKKRSPLTTGVYVKMPPTEPECEKQFQPYFIPIN

SEQ ID NO: 2 - protein, 23 aa, Artificial Sequence
  Synthetic Peptide
YICKVELMYPPPYYLGIGNGTQI

SEQ ID NO: 3 - protein, 6 aa, Artificial Sequence
  Synthetic Peptide
MYPPPY

SEQ ID NO: 4 - protein, 23 aa, Artificial Sequence
  Synthetic Peptide
EVRVTVLRQADSQVTEVCAATYM

SEQ ID NO: 5 - protein, 15 aa, Artificial Sequence
  Synthetic Peptide
GKATEVRVTVLRQAD

SEQ ID NO: 6 - protein, 18 aa, Artificial Sequence
  Synthetic Peptide
VTEVCAATYMMGNELTFL

SEQ ID NO: 7 - protein, 10 aa, Artificial Sequence
  Synthetic Peptide
RGIASFVCEY

SEQ ID NO: 8 - protein, 20 aa, Artificial Sequence
  Synthetic Peptide
SICTGTSSGNQVNLTIQGLR

SEQ ID NO: 9 - protein, 236 aa, Artificial Sequence
  Synthetic Peptide
MQIPQAPWPVVWAVLQLGWRPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTGTIGARRTGQPLKEDPSAVPVFSVDYGELDFQWREKTPEPPVPCVPEQTEYATIVFPSGMGTSSPARRGSADGPRSAQPLRPEDGHCSWPL

SEQ ID NO: 10 - protein, 14 aa, Artificial Sequence
  Synthetic Peptide
SESFVLNWYRMSPS

SEQ ID NO: 11 - protein, 19 aa, Artificial Sequence
  Synthetic Peptide
QTDKLAAFPEDRSQPGQDC

SEQ ID NO: 12 - protein, 21 aa, Artificial Sequence
  Synthetic Peptide
DSGTYLCGAISLAPKAQIKES

SEQ ID NO: 13 - protein, 17 aa, Artificial Sequence
  Synthetic Peptide
KAQIKESLRAELRVTER

SEQ ID NO: 14 - protein, 241 aa, Artificial Sequence
  Synthetic Peptide
MKTLPAMLGTGKLFWVFFLIPYLDIWNIHGKESCDVQLYIKRQSEHSILAGDPFELECPVKYCANRPHVTWCKLNGTTCVKLEDRQTSWKEEKNISFFILHFEPVLPNDNGSYRCSANFQSNLIESHSTTLYVTGKQNELSDTAGREINLVDAHLKSEQTEASTRQNSQVLLSETGIYDNDPDLCFRMQEGSEVYSNPCLEENKPGIVYASLNHSVIGLNSRLARNVKEAPTEYASICVRS

SEQ ID NO: 15 - protein, 16 aa, Artificial Sequence
  Synthetic Peptide
DVQLYIKRQSEHSILA

SEQ ID NO: 16 - protein, 14 aa, Artificial Sequence
  Synthetic Peptide
CSANFQSNLIESHS

SEQ ID NO: 17 - protein, 16 aa, Artificial Sequence
  Synthetic Peptide
RPHVTWCKLNGTTCVK

(19) United States
(12) Patent Application Publication
(10) Pub. No.: US 2018/0256715 A1
(43) Pub. Date: Sep. 13, 2018

(54) ANTI-CTLA-4 BLOCKADE

(71) Applicants: Robert Kirken, El Paso, TX (US); Georgialina Rodriguez, El Paso, TX (US)

(72) Inventors: Robert Kirken, El Paso, TX (US); Georgialina Rodriguez, El Paso, TX (US)

(73) Assignee: THE BOARD OF REGENTS OF THE UNIVERSITY OF TEXAS SYSTEM, Austin, TX (US)

(21) Appl. No.: 15/571,174

(22) PCT Filed: May 13, 2016
(86) PCT No.: PCT/US16/32423

§ 371 Date: Nov. 1, 2017

Related U.S. Application Data
(60) Provisional application No. 62/161,116, filed on May 13, 2015.

(51) Int. Cl. A61K 39/395; A61P 35/00

(52) U.S. Cl. CPC A61K 39/39558; A61K 2039/505; A61P 35/00

(57) ABSTRACT

Embodiments are directed to therapeutic antibodies or antibody fragments that relieve suppression of the immune system, as well therapeutic methods using the same.

### BACKGROUND

The induction of an immune response requires recruitment of appropriate immune cells to the site of a foreign pathogen, e.g., a cancer cell. The immune response involves the interplay of a variety of immune modulatory molecules, which not only control the induction and magnitude of the response but also the production of antibodies and/or the activation of cells that reject tissue and destroy infected and neoplastic cells.

Traditional immunotherapeutic strategies have included (i) the immunization of subjects with inactivated/killed tumor cells or tumor antigens to enhance host immune responses against the tumor, (ii) ex vivo transfection of tumor cells with pro-immune cytokines or costimulatory molecules followed by reinjection of the tumor cells into the host, (iii) systemic administration of cytokines, (iv) nonspecific stimulation of the immune system by local administration of inflammatory substances, (v) adoptive cellular immunotherapy using a host's peripheral blood or tumor infiltrating lymphocytes expanded in culture and reinjected, as well as (vi) passive immunotherapy by administration of monoclonal antibodies that specifically bind cancer cells (Abbas, Cellular and Molecular Immunology, 4th Ed., Saunders, Chapter 17, 2000).

Some tumors actively engage in immune suppression to promote their growth. A variety of tumors are known to either express or to induce the expression of factors that suppress tumor-specific immune responses at the tumor site. Studies have identified a number of tumor-secreted or tumor-associated immune suppressive factors—the inhibition of which may restore normal immune functions and render tumors susceptible to eradication by the host immune system. These tumor-associated factors may not only act at the tumor site to suppress antitumor immunity but may also act systemically to inhibit the ability of tumor antigen encoding vaccines to induce effective antitumor immunity. Neutralizing immune suppressive factors may overcome the tumor-associated immune suppression and allow the development of a productive antitumor immune response. There remains a need for additional reagents and methods for overcoming immune suppressive factors found in the tumor microenvironment and systemically in order to elicit an effective anti-tumor response.

### SUMMARY

Antibody based therapeutics can be used to deplete or activate a biological system, and thus dampen the disease state directly or indirectly by stimulating or relieving suppression of the immune system. One example of the benefit of blocking immune suppression to treat cancer was revealed in studies in mice where the blocking CTLA-4 (using specific antibodies such as ipilimumab) led to the rejection of implanted tumors—a blockade of the negative regulator CTLA-4 was sufficient to allow the immune system to attack cancer cells. The translational significance of these findings became apparent when it was reported that the administration of an anti-human CTLA-4 antibody (ipilimumab) produced a significant increase in survival of patients with metastatic melanoma. This Phase III clinical trial result led to the subsequent FDA approval of this strategy for the treatment of melanoma. More recently, it was shown that the antibody, BMS-936558, targeting the co-inhibitory receptor PD1, which is a CTLA-4 family member, induced significant and durable responses in several types of highly refractory tumors.

Certain embodiments are directed to compositions for reducing or attenuating cancer mediated immune suppression. In certain aspects the composition will comprise an affinity reagent that specifically binds an immune suppressive agent, e.g., CTLA-4 or co-inhibitory receptors (CIR). In certain aspects the affinity reagent neutralizes the immune suppressive agent.

Certain embodiments are directed to an antibody or antibody fragment that specifically binds CTLA-4 (for an example of a CTLA-4 protein having the amino acid sequence MACLGFQRHKAQLNLATRTWPCTLLFFLLFIPVFCKAMHVAQPAVVLASSRGIASFV CEYASPGKATEVRVTVLRQADSQVTEVCAATYMMGNELTFLDDSICTGTSSGNQVN LTIQGLRAMDTGLYICKVELMYPPPYYLGIGNGTQIYVIDPEPCPDSDFLLWILAAVSS GLFFYSFLLTAVSLSKMLKKRSPLTTGVYVKMPPTEPECEKQFQPYFIPIN ((SEQ ID NO:1) see GenBank accession no. AAH74893.1)). In certain aspects the antibody or antibody fragment is a conformation specific antibody. In a further aspect the conformation specific antibody does not bind a linear epitope with a high enough affinity to neutralize or block the activity of CTLA-4. In certain aspects an antibody or antibody fragment can specifically bind 5, 6, 7, 8, 9, or 10 consecutive amino acids of SEQ ID NO:1.

In certain aspects an antibody or antibody fragment can specifically bind the CDR3 loop of the CTLA-4 protein (e.g., residues 134-139 of SEQ ID NO:1). In a further aspect the antibody can specifically bind the amino acid sequence YICKVELMYPPPYYLGIGNGTQI (SEQ ID NO:2). In still a further aspect the antibody can specifically bind the amino acid sequence MYPPPY (SEQ ID NO:3).

In certain aspects an antibody or antibody fragment can specifically bind a first segment of C strands (residues 68-90 of SEQ ID NO:1) of CTLA-4. In a further aspect the antibody specifically binds the amino acid sequence EVRVTVLRQADSQVTEVCAATYM (SEQ ID NO:4).

In certain aspects an antibody or antibody fragment can specifically bind a second segment of C strands (residues 64-78 of SEQ ID NO:1). In a further aspect an antibody specifically binds the amino acid sequence of GKATEVRVTVLRQAD (SEQ ID NO:5).

In certain aspects an antibody or antibody fragment can specifically bind a third segment of the C strand (residues 81-98 of SEQ ID NO:1). In still a further aspect an antibody binds the amino acid sequence VTEVCAATYMMGNELTFL (SEQ ID NO:6).

In certain aspects an antibody or antibody fragment binds to the segment(s) of the CTLA-4 protein that contain the amino acids that form the dimer interface. The dimer interface comprises residues 51, 53, 59, 106, and 117 of SEQ ID NO:1. In a further aspect an antibody specifically binds the amino acid sequence RGIASFVCEY (SEQ ID NO:7) or SICTGTSSGNQVNLTIQGLR (SEQ ID NO:8).

Certain embodiments are directed to affinity reagents that specifically bind and neutralize or block the activity of hPD1 or hBTLA.

In certain aspects an antibody or antibody fragment specifically binds human PD1, which has the amino acid sequence of MQIPQAPWPVVWAVLQLGWRPGWFLDSPDRPWNPPTFSPALLVVTEGDNATFTCSF SNTSESFVLNWYRMSPSNQTDKLAAFPEDRSQPGQDCRFRVTQLPNGRDFHMSVVR ARRNDSGTYLCGAISLAPKAQIKESLRAELRVTGTIGARRTGQPLKEDPSAVPVFSVD YGELDFQWREKTPEPPVPCVPEQTEYATIVFPSGMGTSSPARRGSADGPRSAQPLRPE DGHCSWPL (SEQ ID NO:9). In certain aspects an antibody or antibody fragment specifically binds and neutralizes or blocks the activity of PD1. In a further aspect an antibody or antibody fragment can bind 5, 6, 7, 8, 9, 10 or more consecutive residues of SEQ ID NO:9.

In certain aspects an antibody or antibody fragment specifically binds a segment of the C′, C′C″, FG, or G strand of PD1. In a further aspect an antibody binds the amino acid sequence SESFVLNWYRMSPS (SEQ ID NO:10), QTDKLAAFPEDRSQPGQDC (SEQ ID NO:11), DSGTYLCGAISLAPKAQIKES (SEQ ID NO:12), or KAQIKESLRAELRVTER (SEQ ID NO:13).

In certain aspects an antibody or antibody fragment specifically binds human BTLA, which has the amino acid sequence of MKTLPAMLGTGKLFWVFFLIPYLDIWNIHGKESCDVQLYIKRQSEHSILAGDPFELEC PVKYCANRPHVTWCKLNGTTCVKLEDRQTSWKEEKNISFFILHFEPVLPNDNGSYRC SANFQSNLIESHSTTLYVTGKQNELSDTAGREINLVDAHLKSEQTEASTRQNSQVLLS ETGIYDNDPDLCFRMQEGSEVYSNPCLEENKPGIVYASLNHSVIGLNSRLARNVKEA PTEYASICVRS (SEQ ID NO:14). In certain aspects an antibody or antibody fragment specifically binds and neutralizes or blocks the activity of BTLA. In a further aspect an antibody or antibody fragment can bind 5, 6, 7, 8, 9, 10 or more consecutive residues of SEQ ID NO:14.

In certain aspects an antibody or antibody fragment specifically binds a segment of the A′ strand, G′ strand, or CC′loop of BTLA. In a further aspect an antibody binds the amino acid sequence DVQLYIKRQSEHSILA (SEQ ID NO:15), CSANFQSNLIESHS (SEQ ID NO:16), or RPHVTWCKLNGTTCVK (SEQ ID NO:17).

The term “antibody” or “immunoglobulin” is used to include intact antibodies and binding fragments/segments (functional fragments) thereof. “Functional fragments” of such antibodies comprise portions of intact antibodies that retain a similar antigen-binding specificity to the parent antibody molecule. For example, functional fragments can comprise at least the CDRs of either the heavy chain or light chain variable region. Functional fragments can also comprise the heavy chain or light chain variable region, or sequences that are substantially similar to the heavy or light chain variable region. Further suitable functional fragments include, without limitation, antibodies with multiple epitope specificity, bispecific antibodies, diabodies, and single-chain molecules, as well as Fab, F(ab′)2, Fd, Fabc, and Fv molecules, single chain (Sc) antibodies (also called ScFv), individual antibody light chains, individual antibody heavy chains, chimeric fusions between antibody chains and other molecules, heavy chain monomers or dimers, light chain monomers or dimers, dimers consisting of one heavy and one light chain, and the like. All antibody isotypes can be used to produce functional fragments of the antibodies herein. Functional fragments can be recombinantly or synthetically produced, with natural or unnatural nucleic acid or amino acid molecules.

The antibodies or functional fragments thereof of the disclosed subject matter can be generated from any species. The antibodies or functional fragments thereof described herein can be labeled or otherwise conjugated to various chemical or biomolecule moieties, for example, for therapeutic or diagnostic or detection or treatment applications. The moieties can be cytotoxic, for example, bacterial toxins, viral toxins, radioisotopes, and the like. The moieties can be detectable labels, for example, fluorescent labels, radiolabels, biotin, and the like, which are known in the art.

As used herein, the term “monoclonal antibody” refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single epitope. Furthermore, in contrast to conventional (polyclonal) antibody preparations, which typically include different antibodies directed against different determinants (epitopes), each monoclonal antibody is directed against a single determinant on the antigen. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by the hybridoma method first described by Kohler, et al., Nature 256:495 (1975), or may be made by recombinant DNA methods (see, e.g., U.S. Pat. No. 4,816,567). The “monoclonal antibodies” may also be isolated from phage antibody libraries using the techniques described in Clackson et al., Nature 352:624-628 (1991) and Marks et al., J. Mol. Biol. 222:581-597 (1991), for example.

The term “human sequence antibody” or “human antibody” includes antibodies having variable and constant regions (if present) derived from human germline immunoglobulin sequences. The human sequence antibodies of the invention may include amino acid residues not encoded by human germline immunoglobulin sequences (e.g., mutations introduced by random or site-specific mutagenesis in vitro or by somatic mutation in vivo). However, the term “human sequence antibody”, as used herein, is not intended to include antibodies in which CDR sequences derived from the germline of another mammalian species, such as a mouse, have been grafted onto human framework sequences (i.e., humanized antibodies).

The antibodies or functional fragments thereof described herein have binding affinities in M for their respective targets that include a dissociation constant (K_{D}) of less than 1×10⁻². In some embodiments, the K_{D} is less than 1×10⁻³. In other embodiments, the K_{D} is less than 1×10⁻⁴. In some embodiments, the K_{D} is less than 1×10⁻⁵. In still other embodiments, the K_{D} is less than 1×10⁻⁶, 1×10⁻⁷, 1×10⁻⁸, 1×10⁻⁹, 1×10⁻¹⁰, 1×10⁻¹¹, 1×10⁻¹², 1×10⁻¹³, 1×10⁻¹⁴, or 1×10⁻¹⁵.

As used herein, the term “antigen” is a molecule capable of being bound by an antibody or T-cell receptor. An antigen is additionally capable of inducing a humoral immune response and/or cellular immune response leading to the production of B- and/or T-lymphocytes. The structural aspect of an antigen, e.g., three-dimensional conformation or modification (e.g., phosphorylation), giving rise to a biological response is referred to herein as an “antigenic determinant” or “epitope.” B-lymphocytes respond to foreign antigenic determinants via antibody production, whereas T-lymphocytes are the mediator of cellular immunity. Thus, antigenic determinants or epitopes are those parts of an antigen that are recognized by antibodies, or in the context of an MHC, by T-cell receptors. An antigenic determinant need not be a contiguous sequence or segment of protein and may include various sequences that are not immediately adjacent to one another. In certain embodiments, binding moieties other than antibodies and be engineered to specifically bind to an antigen, e.g., aptamers, avimers, and the like.

Moieties of the invention, such as polypeptides, peptides, antibodies, antigens, or immunogens, may be conjugated or linked covalently or noncovalently to other moieties such as adjuvants, proteins, peptides, supports, fluorescence moieties, or labels. The term “conjugate” or “immunoconjugate” is broadly used to define the operative association of one moiety with another agent and is not intended to refer solely to any type of operative association, and is particularly not limited to chemical “conjugation.”

The phrase “specifically binds” or “specifically immunoreactive” to a target refers to a binding reaction that is determinative of the presence of the molecule in the presence of a heterogeneous population of other biologics. Thus, under designated immunoassay conditions, a specified molecule binds preferentially to a particular target and does not bind in a significant amount to other biologics present in the sample. Specific binding of an antibody to a target under such conditions requires the antibody be selected for its specificity to the target. A variety of immunoassay formats may be used to select antibodies specifically immunoreactive with a particular protein. For example, solid-phase ELISA immunoassays are routinely used to select monoclonal antibodies specifically immunoreactive with a protein. See, e.g., Harlow and Lane, Antibodies: A Laboratory Manual, Cold Spring Harbor Press, 1988, for a description of immunoassay formats and conditions that can be used to determine specific immunoreactivity.

The terms “treating” or “treatment” refer to any success or indicia of success in the attenuation or amelioration of an injury, pathology or condition, including any objective or subjective parameter such as abatement, remission, diminishing of symptoms or making the injury, pathology, or condition more tolerable to the patient, slowing in the rate of degeneration or decline, making the final point of degeneration less debilitating, improving a subject's physical or mental well-being, or prolonging the length of survival. The treatment or amelioration of symptoms can be based on objective or subjective parameters; including the results of a physical examination, neurological examination, and/or psychiatric evaluations.

The phrases “treating cancer” and “treatment of cancer” mean to decrease, reduce, or inhibit the replication of cancer cells; decrease, reduce or inhibit the spread (formation of metastases) of cancer; decrease tumor size; decrease the number of tumors (i.e. reduce tumor burden); lessen or reduce the number of cancerous cells in the body; prevent recurrence of cancer after surgical removal or other anti-cancer therapies; or ameliorate or alleviate the symptoms of the disease caused by the cancer.

The terms “inhibiting,” “reducing,” or “prevention,” or any variation of these terms, when used in the claims and/or the specification includes any measurable decrease or complete inhibition to achieve a desired result.

“Effective amount” and “therapeutically effective amount” are used interchangeably herein, and refer to an amount of an antibody or functional fragment thereof, as described herein, effective to achieve a particular biological or therapeutic result such as, but not limited to, the biological or therapeutic results disclosed herein. A therapeutically effective amount of the antibody or antigen-binding fragment thereof may vary according to factors such as the disease state, age, sex, and weight of the individual, and the ability of the antibody or functional fragment thereof to elicit a desired response in the individual. Such results may include, but are not limited to, the treatment of cancer, as determined by any means suitable in the art.

The term “isolated” can refer to a nucleic acid or polypeptide that is substantially free of cellular material, bacterial material, viral material, or culture medium (when produced by recombinant DNA techniques) of their source of origin, or chemical precursors or other chemicals (when chemically synthesized). Moreover, an isolated compound refers to one that can be administered to a subject as an isolated compound; in other words, the compound may not simply be considered “isolated” if it is adhered to a column or embedded in an agarose gel. Moreover, an “isolated nucleic acid fragment” or “isolated peptide” is a nucleic acid or protein fragment that is not naturally occurring as a fragment and/or is not typically in the functional state.

Other embodiments of the invention are discussed throughout this application. Any embodiment discussed with respect to one aspect of the invention applies to other aspects of the invention as well and vice versa. Each embodiment described herein is understood to be embodiments of the invention that are applicable to all aspects of the invention. It is contemplated that any embodiment discussed herein can be implemented with respect to any method or composition of the invention, and vice versa. Furthermore, compositions and kits of the invention can be used to achieve methods of the invention.

The use of the word “a” or “an” when used in conjunction with the term “comprising” in the claims and/or the specification may mean “one,” but it is also consistent with the meaning of “one or more,” “at least one,” and “one or more than one.”

Throughout this application, the term “about” is used to indicate that a value includes the standard deviation of error for the device or method being employed to determine the value.

The use of the term “or” in the claims is used to mean “and/or” unless explicitly indicated to refer to alternatives only or the alternatives are mutually exclusive, although the disclosure supports a definition that refers to only alternatives and “and/or.”

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### DESCRIPTION OF THE DRAWINGS

The following drawings form part of the present specification and are included to further demonstrate certain aspects of the present invention. The invention may be better understood by reference to one or more of these drawings in combination with the detailed description of the specification embodiments presented herein.

FIG. 1. Illustration of one method for production of monoclonal antibodies toward the Extracellular Domain (ECD) of CTLA4. Antigen, ECD, is injected into Balb/c mice. The natural immune system of the mouse develops antibodies against ECD. B-cell rich spleen cells are harvested and fused with immortal SP20 myeloma cells using polyethylene glycol (PEG). Cells are selected based on their ability to make the desired antibody and clones are propagated. Hybridoma clones can be grown in mass to produce large amounts of antibody which can then be purified or injected into a balb/c mouse and the resulting antibody rich ascetic fluid collected.

FIG. 2. Illustrates one methods for large scale purification for the isolation of monoclonal antibodies. Antibody rich hybridoma media is run through a protein G sepharose bead column. Antibody bound beads are washed to increase purity and eluted to release 8-10 mg of purified monoclonal antibody.

FIG. 3. Anti-CTLA4 hybridoma cell lines produced from immunization with (A) ECD or (B) peptide sequence were isotyped.

FIG. 4. ECD derived monoclonal antibodies were analyzed by ELISA. 96-well plates were coated with ECD peptide and antibodies were tested for their ability to recognize the target at the listed dilutions. Titer refers to the functional dilution of the antibody necessary to achieve the desired detection range (signal/blank>=2.1).

FIG. 5. Peptide derived monoclonal antibodies were analyzed by ELISA. 96-well plates were coated with epitope sequence and antibodies were tested for their ability to recognize the target. Titer refers to the functional dilution of the antibody necessary to achieve the desired detection range (signal/blank>=2.1).

FIG. 6. ECD and control (Ctrl) protein were spotted onto PVDF membrane in decreasing concentrations from left to right. Western blot analysis was performed with monoclonal antibodies at 1:1000 dilution. Representative dot blots shown.

FIG. 7A-7B. Detection of CTLA4 in Jurkat cell line by Flow Cytometry. Jurkat cells transfected with human CTLA-4 was stained with either (A) ECD derived monoclonal antibodies or (B) peptide derived monoclonal antibodies followed by Alexa Fluor conjugated anti-mouse IgG.

FIG. 8. In Vitro analysis of anti-CTLA4 mABs. Human Jurkat cells were either unactivated (lane a) or activated by the addition of 10 μg anti-CD3 mAB plus 250 ng purified B7.1 protein (lane b-j). Additionally cells (lanes c-j) received 150 ng recombinant human CTLA-4 protein (rhCTLA4). 10 μg of the indicated antibodies were used to neutralize the suppressive effects of rhCTLA4 (lanes d-j) for 24-hours in the presence of activating proteins mentioned above. Cell media was harvested and assayed for IL-2 secretion by Elisa.

FIG. 9. In Vivo Tumor Suppression by Novel Monoclonal Antibodies. Murine EMT-6/P cells were implanted s.c. in female Balb/c mice. Therapies began when tumors were 50 mm³; Mice received control PBS (i.p.), CTLA-4 antibodies or a combination of anti-CTLA4 (4D11A8 and 5D3H5). One group received anti-CTLA4 (9H10) therapy as a first line treatment and then a second line therapy consisting of clone 5D3H5 antibody.

FIG. 10. PD-1 Indirect Elisa to Determine Antibody Titer.

FIG. 11A-11C. Purified PD1 Antibodies Separated by SDS-PAGE & Stained with Coomassie Brilliant Blue.

FIG. 12. Dot Blot Testing of PD-1.

FIG. 13. Flow Cytometry Analysis of PD-1 Clones. (A) Cells only. (B) Secondary. (C) Anti-PD1 commercial. (D) Clone 2A6E6. (E) Clone 2H11D3. (F) Clone 3H3F4. (G) Clone 5B3D5. (H) Clone 1007D3.

FIG. 14. BTLA Indirect Elisa to Determine Antibody Titer.

FIG. 15. Dot Blot Testing of BTLA Clones.

### DESCRIPTION

Immune tolerance allows cancer cells to avoid recognition and elimination by the host immune system. Embodiments of the invention described herein provides methods and compositions for neutralizing or blocking immune suppression. In certain aspects affinity reagents to components of the immune suppression pathways that reduce, attenuate, or block immune suppression are produced and formulated for administration to a patient in need.

CTLA4 is a member of the CD28 receptor family along with the co-inhibitory receptors (CIR) PD1 and BTLA, which function by recruiting phosphatases to reverse events activated by phosphorylation. Like the ligands of CD28 and CTLA-4, PD1 ligand (PD-L1 and PD-L2) are B7 family proteins comprised of tandem V-set and C1-set IgSF domains. In addition to PD-1, PD-L1 binds B7-1, one of the ligands of CD28 and CTLA-4, potentially interlocking the PD-1 and CD28/CTLA-4 signaling pathways. Structures of mouse PD-1 complexed with human PD-L1 and mouse PD-L2 revealed that these proteins interact largely orthogonally via their GFCC′C″ β-sheets.

Embodiments described herein are directed to antibodies or antibody fragments and methods of using the same. Certain aspects are directed to conformation specific or monoclonal antibodies toward cytotoxic T-lymphocyte associated protein-4 (CTLA4) or other CIRs (e.g., PD1 and BTLA), to activate the natural immune response and dampen the progression of cancer directly or indirectly. Thus, cancer can be treated with the administration of such antibodies promoting the death of tumor cells. In certain aspects a conformation specific antibody binds a protein in its folded conformation and does not bind a protein that has been denatured or unfolded in the region the antibody binds.

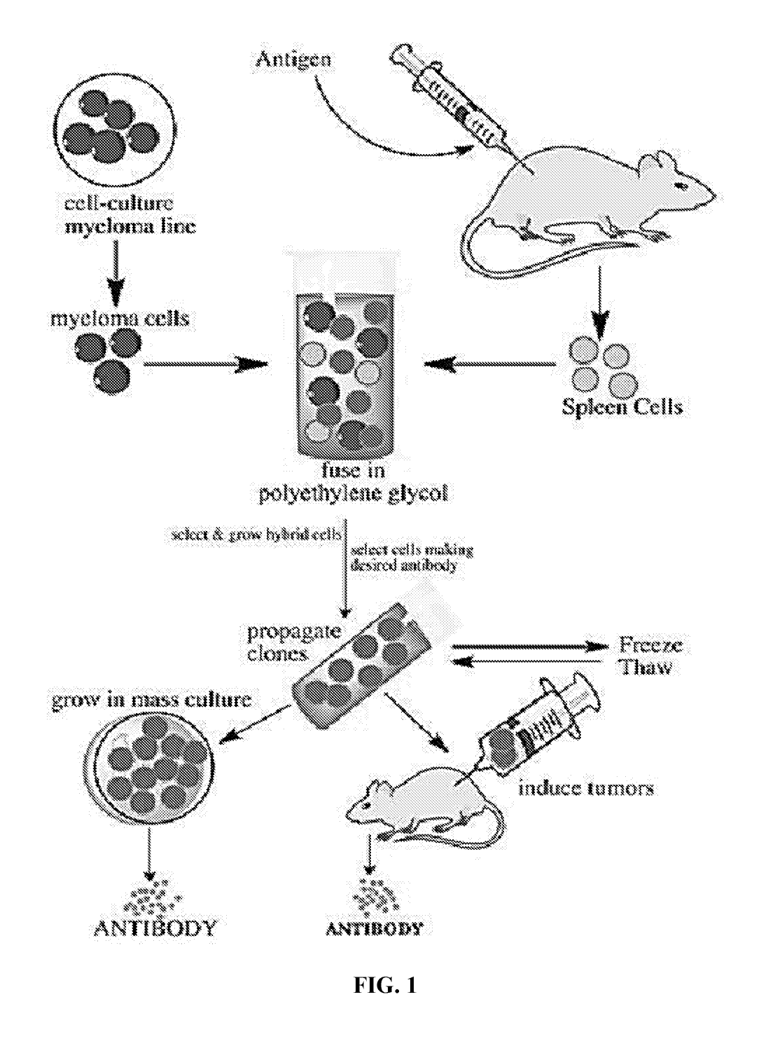

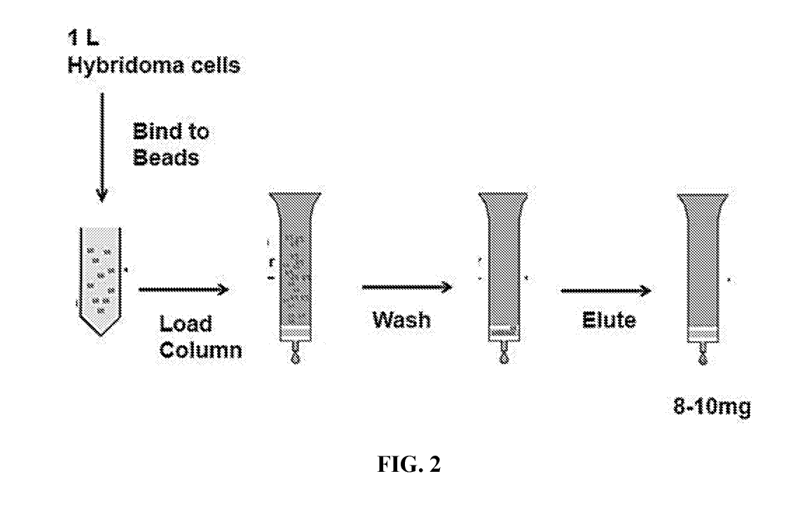

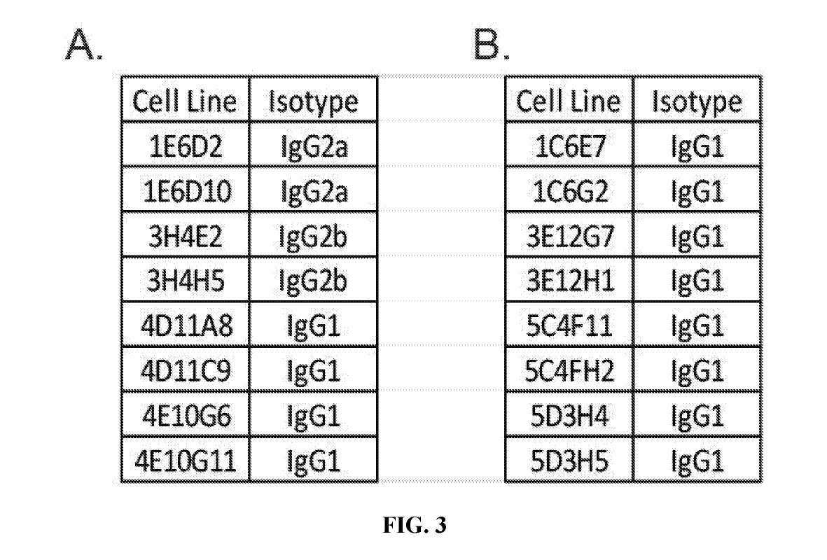

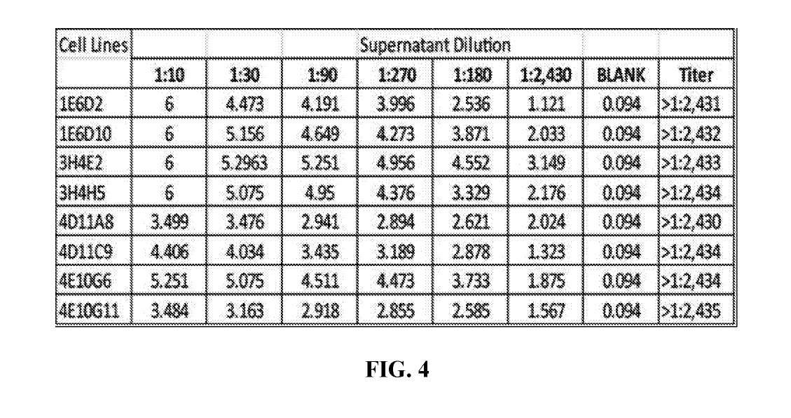

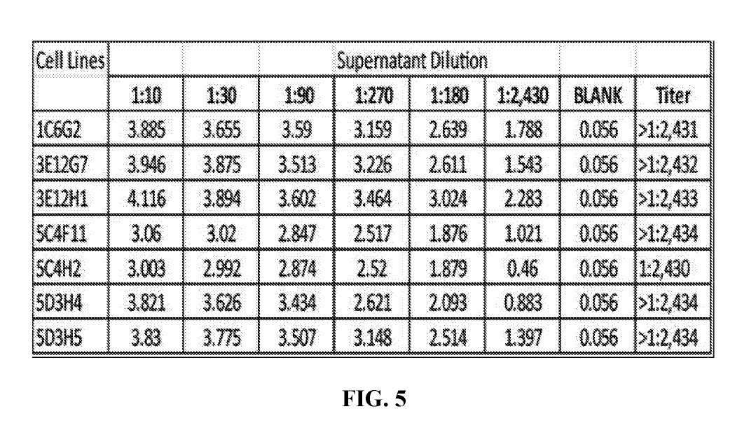

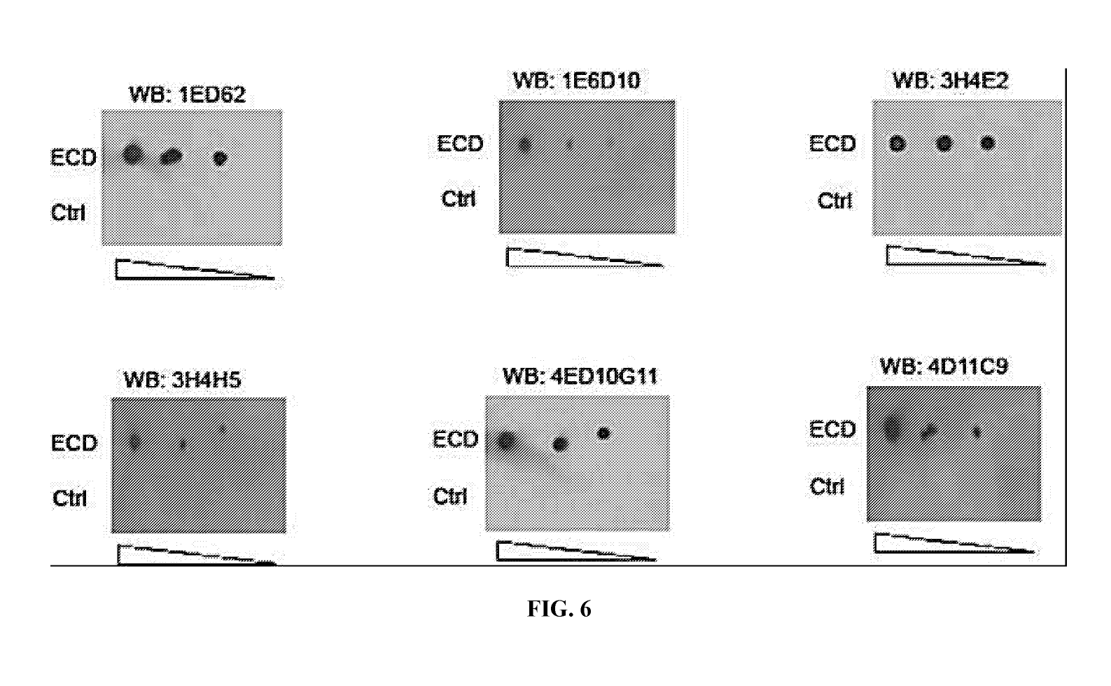

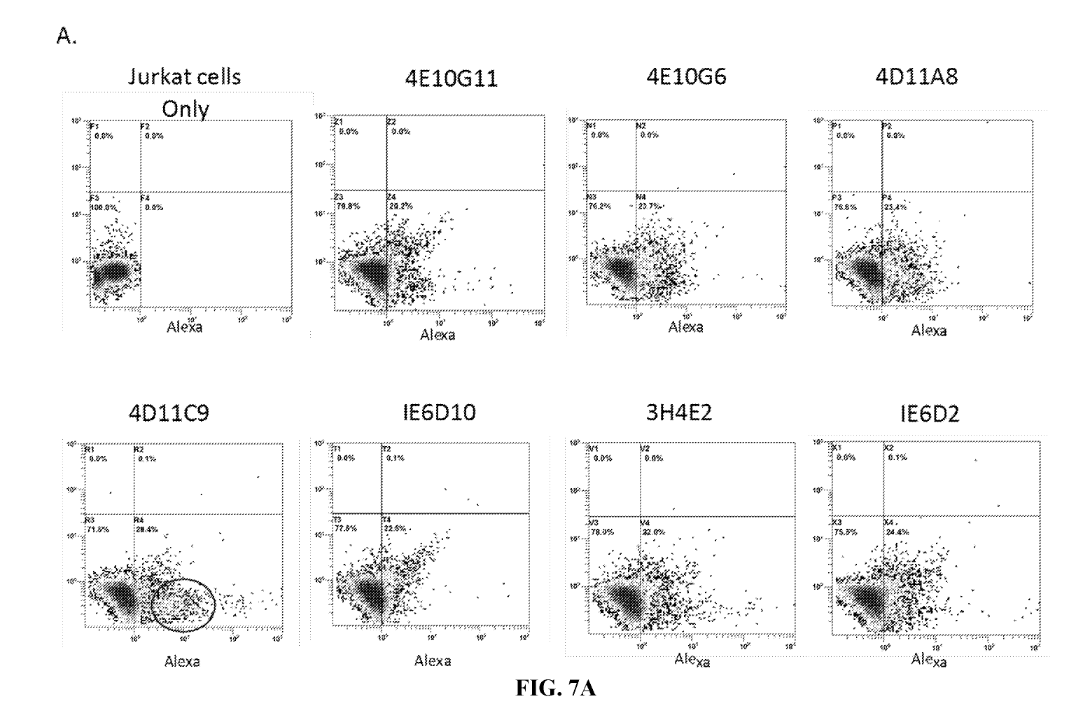

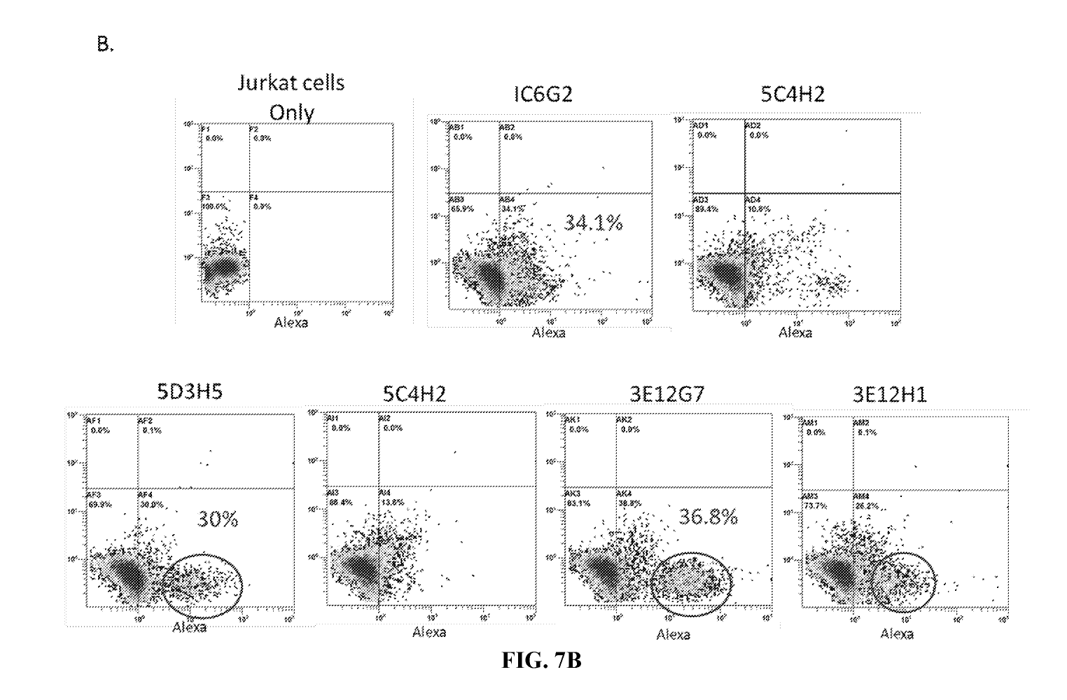

Confirmation specific antibodies can be produced by immunizing with whole proteins or appropriately folded fragments, e.g., extracelluar domain (ECD) and the like (FIG. 1), Conformation specific antibodies bind to secondary or tertiary structure, as compared to linear epitopes (peptides) to develop therapeutic monoclonal antibodies.

Certain embodiments are directed to the treatment of cancer by administering an anti-CIR blockade that promotes the death of tumor cells. The anti-CIR blockade can comprise one or more antibodies. The antibodies can be conformation specific antibodies, monoclonal antibodies, or recombinant antibodies. In certain aspects the antibody, upon binding its target will disrupt the target's ligand binding interface. In any event, regardless of the mechanism the antibodies will neutralize or block the activity of the target to a sufficient degree to impart a therapeutic response.

In certain aspects antibodies can be produced based on the functional and structural domains of the CIR protein(s). Various domains of the targets are used to generate antibodies with therapeutic activity. In certain aspects the following domains were targeted:

CTLA4 is a member of the immunoglobulin superfamily, which is expressed on the surface of Helper T cells and transmits an inhibitory signal to T cells. CTLA-4 is similar to the T-cell co-stimulatory protein, CD28, and both molecules bind to CD80 and CD86, also called B7-1 and B7-2 respectively, on antigen-presenting cells. CTLA-4 transmits an inhibitory signal to T cells, whereas CD28 transmits a stimulatory signal.

For CTLA-4 the anti-CDR3 loop (MYPPPY) was targeted, in particular the amino acid sequence YICKVELMYPPPYYLGIGNGTQI. Another region of CTLA-4 targeted is the C strands, in particular the amino acid sequence EVRVTVLRQADSQVTEVCAATYM, or GKATEVRVTVLRQAD. Still another segment targeted is the C′ strand, in particular the amino acid sequence VTEVCAATYMMGNELTFL. In certain aspects the CTLA-4 dimer interface (comprising residues 51, 53, 59, 106, and 117 of SEQ ID NO:1) is targeted.

These sites were selected, in part, for disrupting the interaction of B7.1 and/or B7.2 with CTLA-4 and are based on the structure for CTLA-4/B7.2. Structural studies have identified the binding interface regions for CTLA-4 and B7.2 monomers. The interface is formed by residues from the CDR3, C, and C′ strands on hCTLA-4 and the concave surface on B7.2 (Schwartz et al. Letters to Nature, 410:604-07, 2001).

Sequence alignment of the CTLA-4 and CD28 family residues involved in the CTLA/B7.2 binding site shows greater than 50% conservation across human, murine, rat and rabbit. Analysis of the CTLA-4 and sB7.1 receptor-ligand binding interface shows that the CDR3 loop (MYPPPY) of hCTLA-4 is buried in the concave depression of sB7.1. The ribbon diagram of CTLA-4 dimer shows that the interface is made up of residues from C-terminal to G strand, centered around the A′ strand.

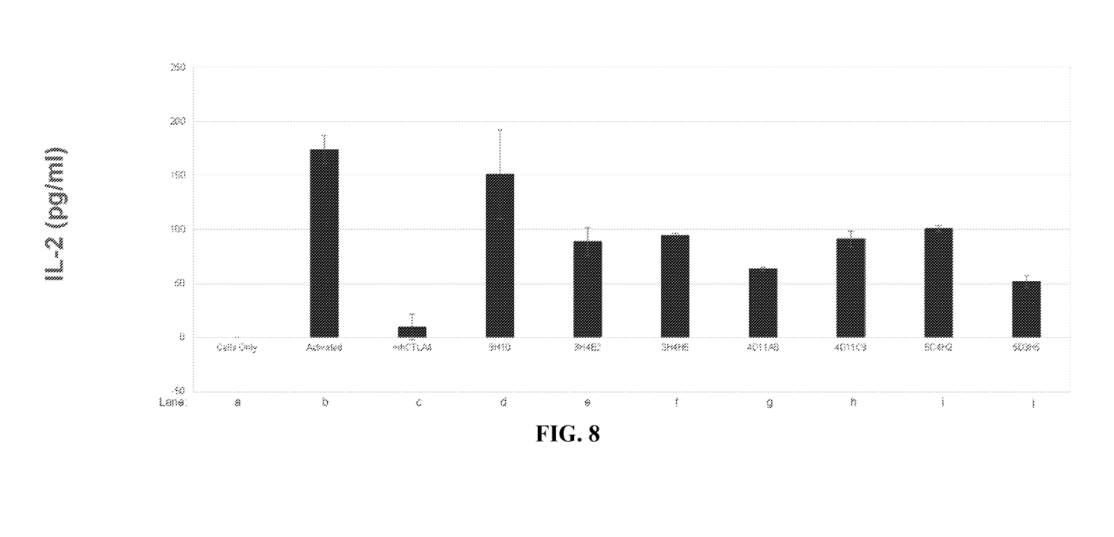

To determine the effectiveness of the monoclonal antibodies directed toward human CTLA-4, human Jurkat leukemia cells, which produce and secrete IL-2, represents a marker of T-cell activation. A test was performed to determine the in vitro neutralization capabilities of these CTLA4 monoclonal antibodies. In FIG. 8, human Jurkat cells responded favorably to activation with anti-CD3 and B7.1 as well as inhibitory signals driven by the addition of recombinant human CTLA-4 (rhCTLA-4) as measured by IL2 release. Following the addition of the novel anti-CTLA4 monoclonal antibodies, the various clones were competent to inhibit CTLA-4 activity: 3H4E2, 3H4H5, 4D11C9 and 5C4H2 quite effectively. For this this assay, the latter clones showed neutralizing capability similar to a commercially available 9H10 antibody. Clones 4D11C9 and 5D3H5 were also effective.

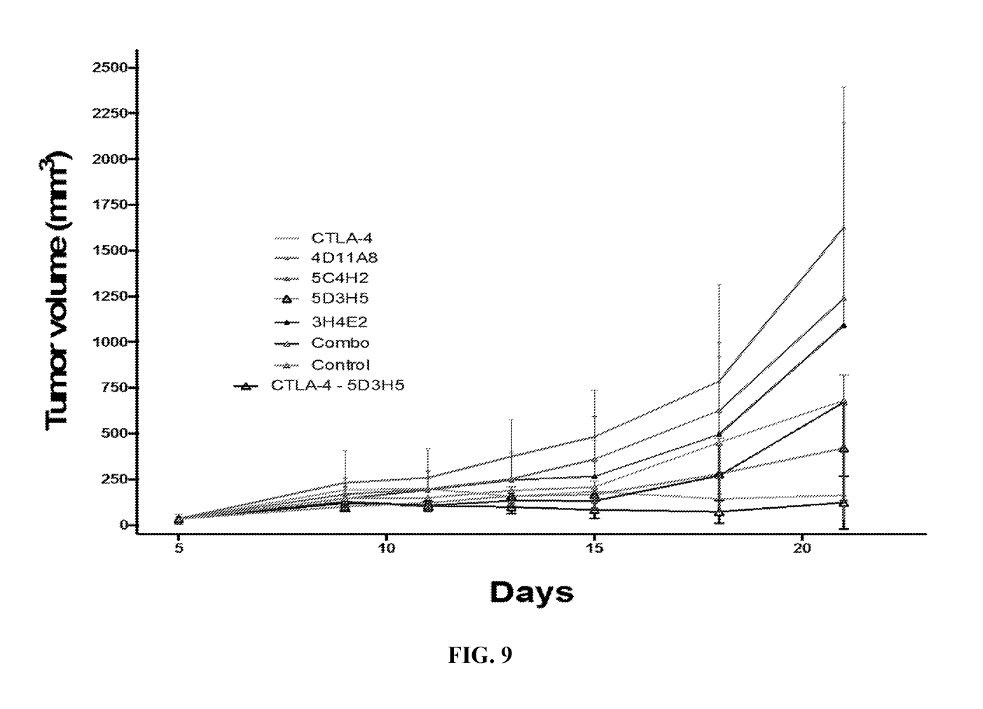

Based on these results clones 4D11A8, 5C4H2, 5D3H5 and 3H4E2 were chosen for in vivo screening and display of anti-tumor activity. Briefly, female Balb/c mice were injected subcutaneously with mouse EMT-6/P cells. Treatment began once tumors reached 50 mm³ following intraperitoneally (i.p.) delivery. Mice that received a control treatment (PBS) showed rapid tumor growth as compared to clones 4D11A8, 5C4H2 or 3H4E2. In comparison, mice treated with anti-CTLA-4 (9H10), clone 5D3H5 or a combination of 5D3H5 plus 4D11A8 responded to therapy (FIG. 9). One group received anti-CTLA-4 (9H10) as a first line treatment followed by 5D3H5 given repeatedly for 20 days displayed similar effects to 9H10 therapy alone. Many of the mice that received clone 5D3H5 or a combination treatment resisted tumor growth during the first 14 days.

Certain embodiments are directed to antibodies that specifically bind CIRs PD1 or BTLA. PD1 (Programmed cell death protein 1 or CD279) is a cell surface receptor that belongs to the immunoglobulin superfamily and is expressed on T cells and pro-B cells. PD-1 binds two ligands, PD-L1 and PD-L2. PD-1 and its ligands play a role in down regulating the immune system by preventing the activation of T-cells. BTLA (B- and T-lymphocyte attenuator or CD272) is induced during activation of T cells, and BTLA remains expressed on Th1 cells but not Th2 cells. Like PD1 and CTLA4, BTLA interacts with a B7 homolog, B7H4. However, unlike PD-1 and CTLA-4, BTLA displays T-Cell inhibition via interaction with tumor necrosis family receptors (TNF-R), not just the B7 family of cell surface receptors. The PD1 and BTLA antibodies disrupt co-inhibitory receptor/ligand binding interface and neutralize or block the activity of PD1 or BTLA, respectively.

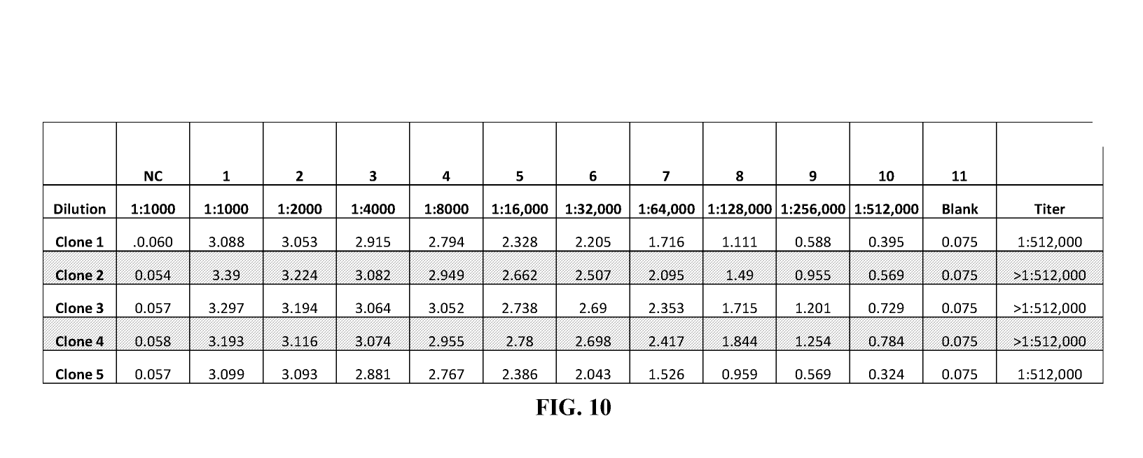

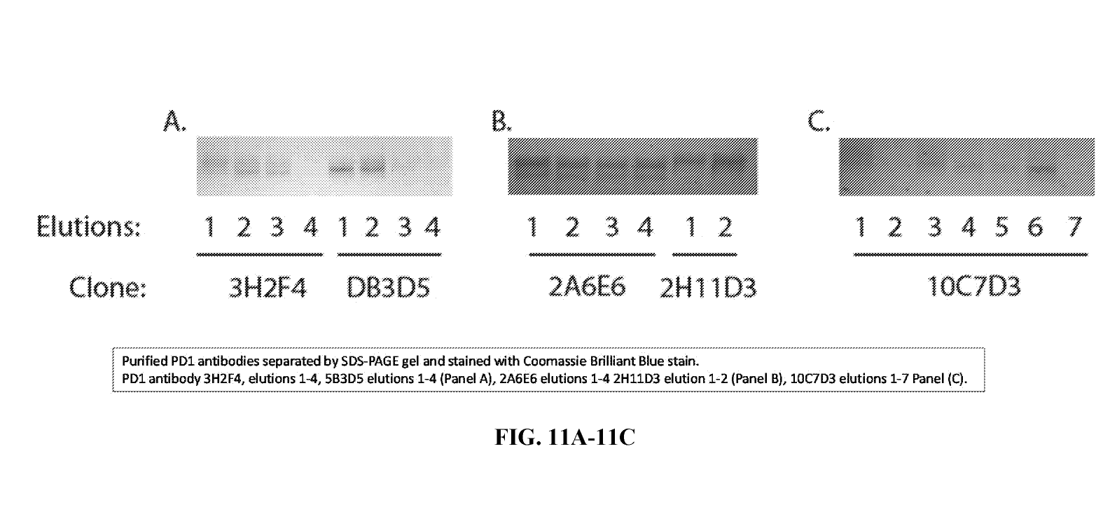

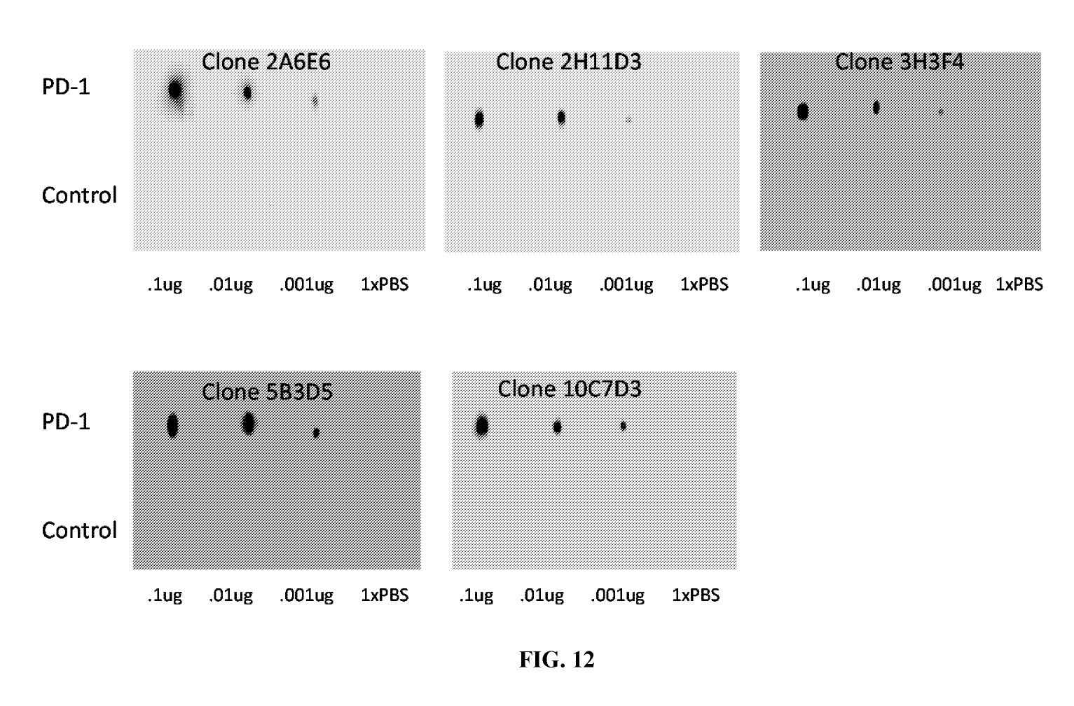

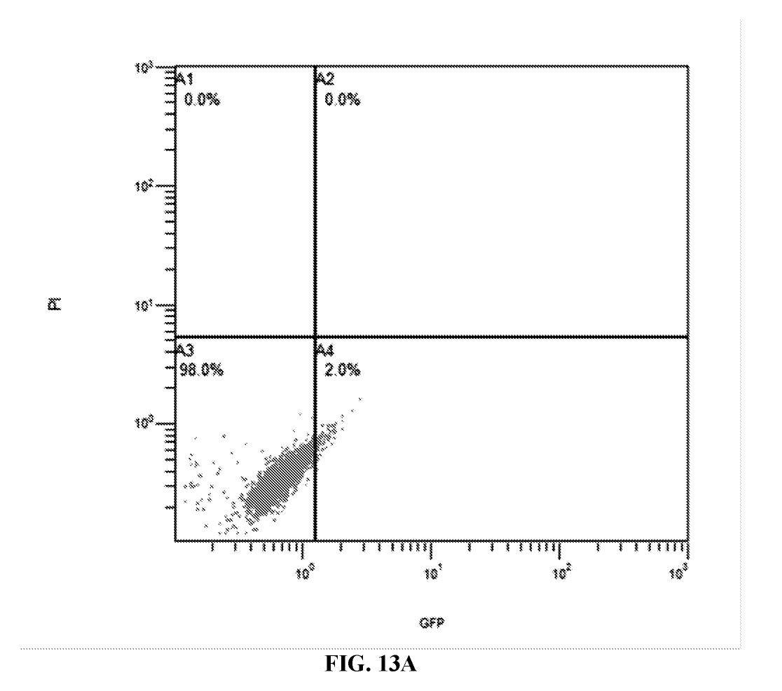

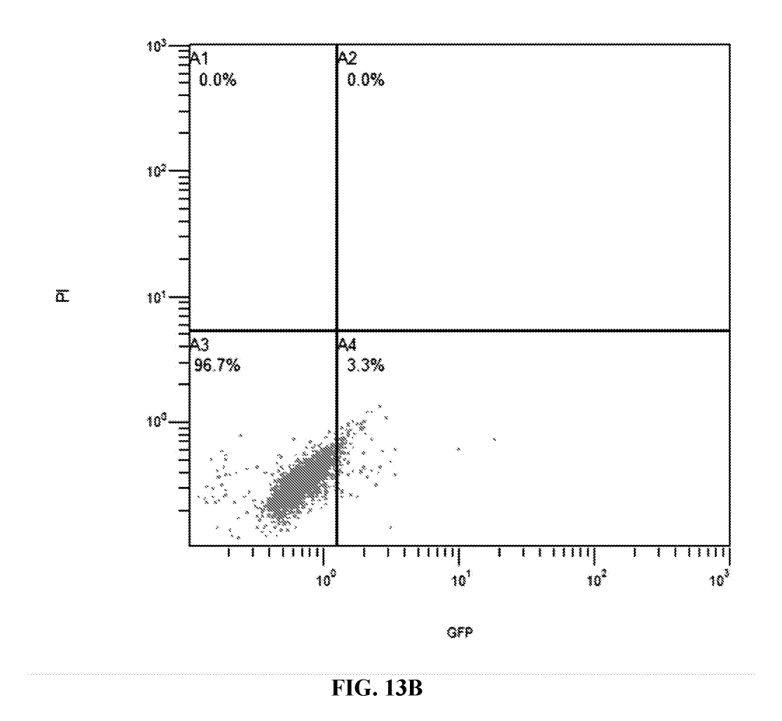

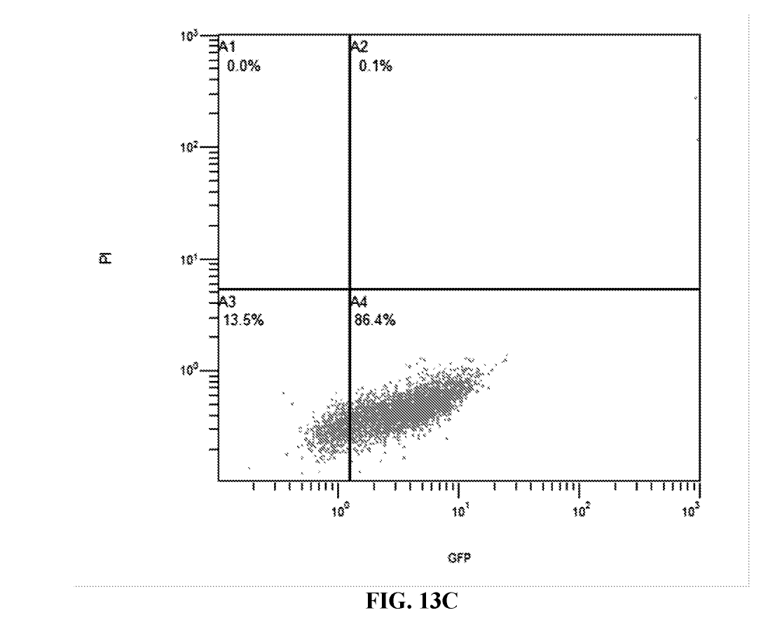

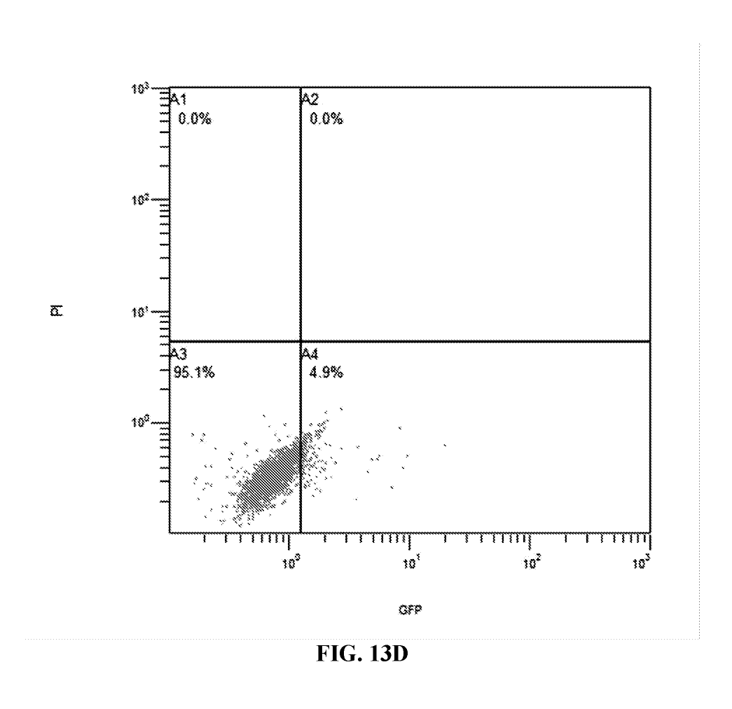

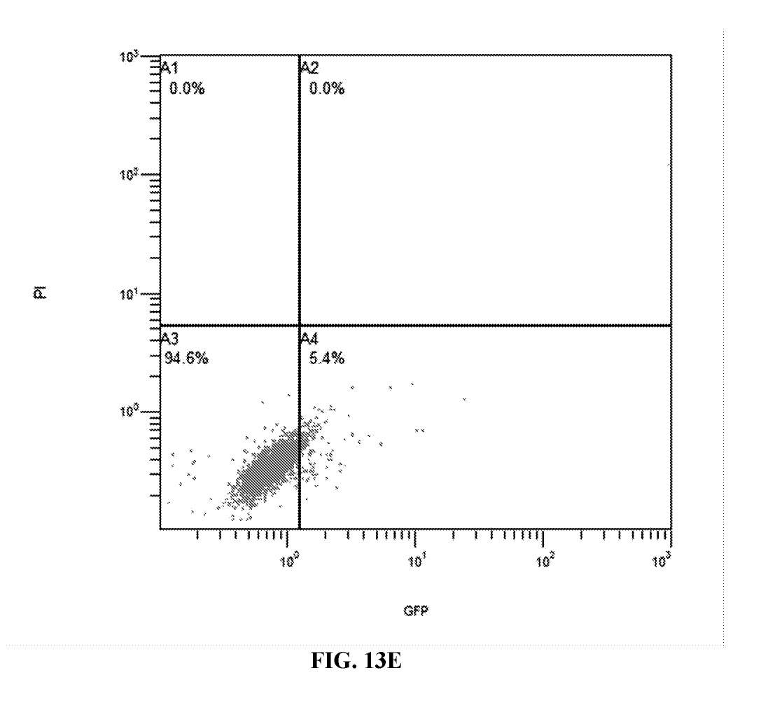

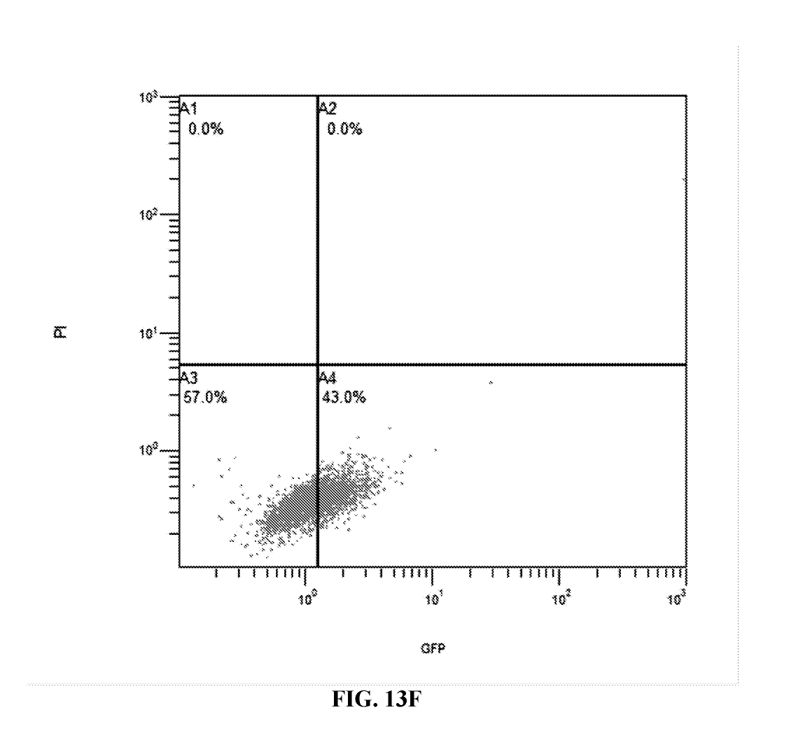

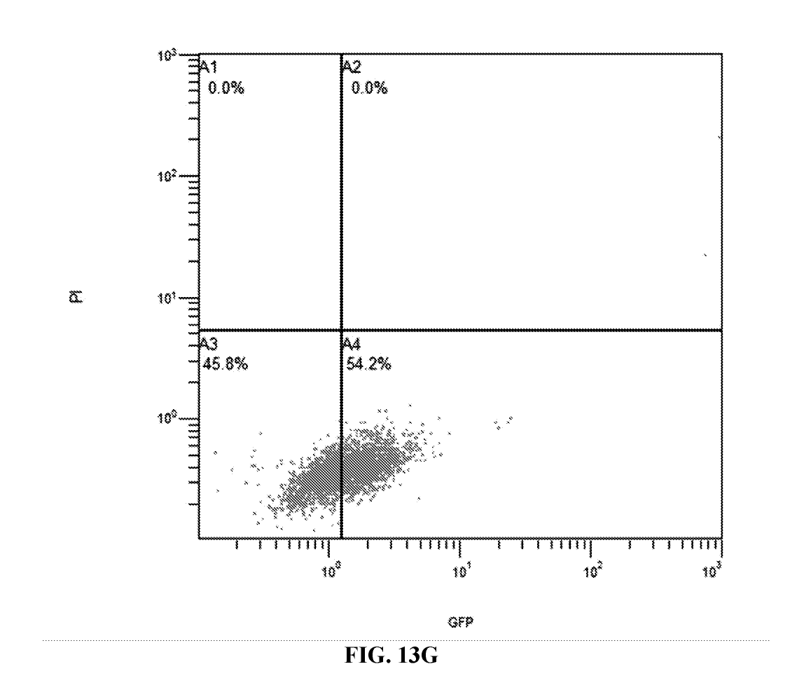

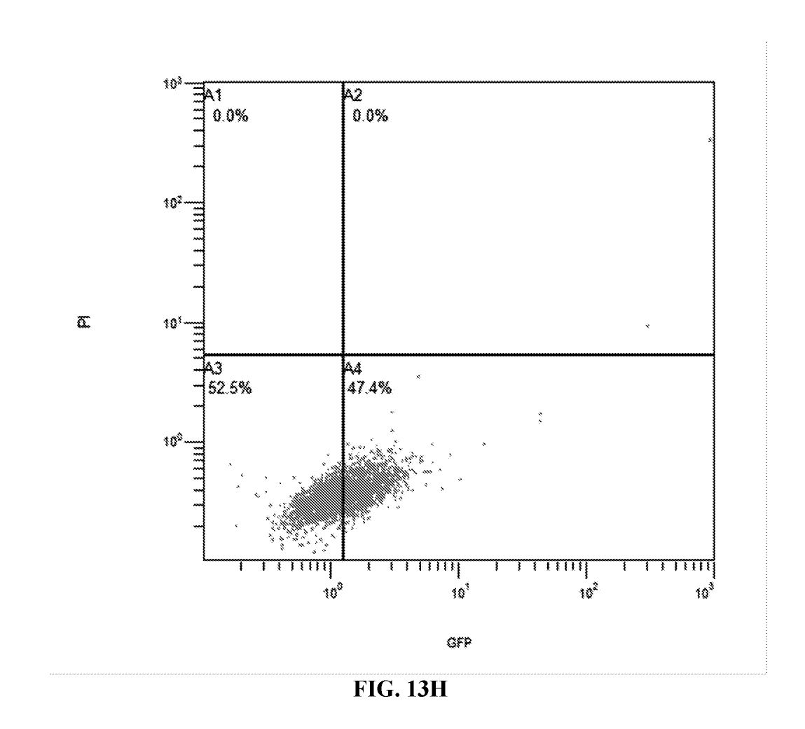

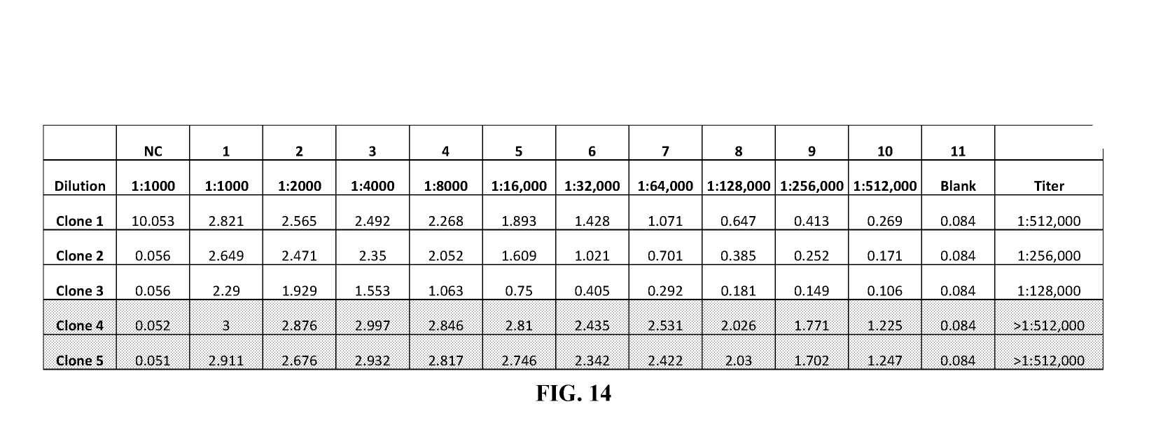

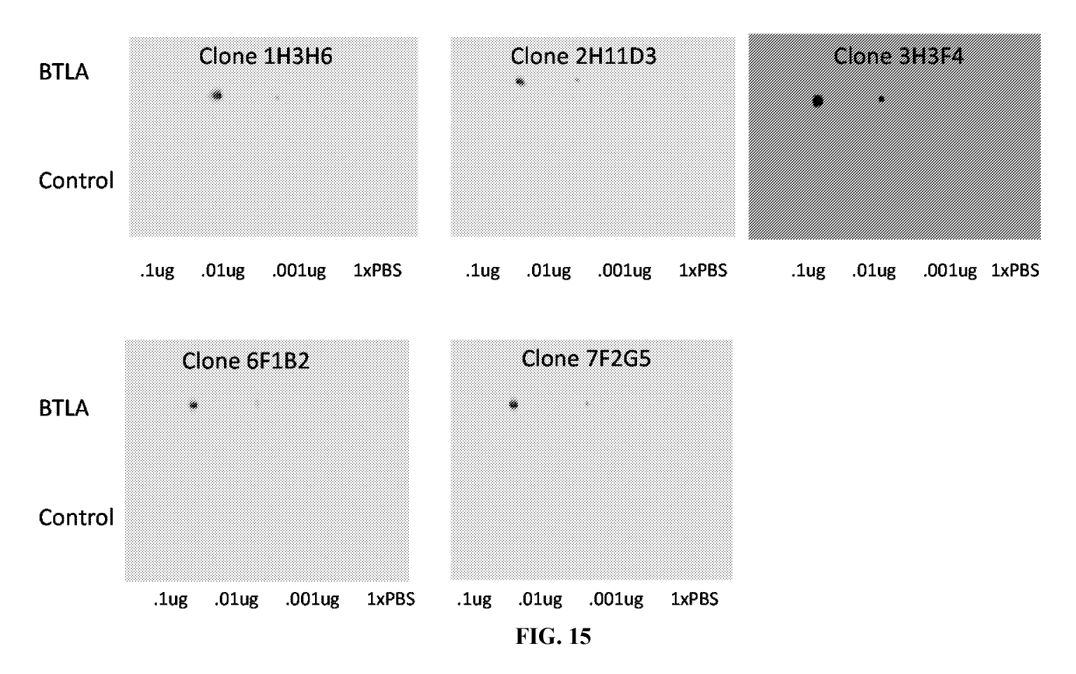

Antibodies to PD1 (see FIG. 10 to FIG. 14) and BTLA (see FIG. 14 and FIG. 15) are developed based on the functional and structural domains of the CIR proteins. The following domains were targeted to develop therapeutic PD1 and BTLA antibodies:

PD1 antibodies are developed using the amino acid sequence of the C′ strand, in particular SESFVLNWYRMSPS. The C′C″ strand is also targeted, in particular QTDKLAAFPEDRSQPGQDC. Another target of PD1 is the FG strand, in particular DSGTYLCGAISLAPKAQIKES. The G strand is also targeted, in particular KAQIKESLRAELRVTER. Residues were identified from the GFC′C″ strands and C′C″, and FG loops of PD-1 contribute to the binding interface of PDL2.

BTLA antibodies are developed using the amino acid sequence of the A′ strand, in particular DVQLYIKRQSEHSILA. G° strand CSANFQSNLIESHS. CC′ loop, RPHVTWCKLNGTTCVK. These sites were selected based on the information regarding BTLA/ligand binding. Crystollography coupled to mutagenesis studies show that strands A′ and G° are part of the BTLA/ligand binding interface. Unlike other CD28 family members, the BTLA binding surface is located along the edge of the I-set Ig domain.

In addition to monoclonal antibodies toward the above CIRs, the inventors are investigating the blockade of BTLA, a CD28 receptor family member expressed at high levels on tumor-specific CTLs that inhibits T cell function upon its engagement by tumor-expressed HVEM. Thus, BTLA blockade may potentially improve T cell antitumor immunity.

Pharmaceutical Formulations and Administration.

In certain embodiments, the invention also provides compositions comprising one or more anti-cancer agents, e.g., therapeutic antibodies, with one or more of the following: a pharmaceutically acceptable diluent; a carrier; a solubilizer; an emulsifier; a preservative; and/or an adjuvant. Such compositions may contain an effective amount of at least one anti-cancer agent. Thus, the use of one or more anti-cancer agents that are provided herein in the preparation of a pharmaceutical composition of a medicament is also included. Such compositions can be used in the treatment of a variety of cancers. In certain embodiments the treatment is for leukemia or breast cancer.

The anti-cancer agents may be formulated into therapeutic compositions in a variety of dosage forms such as, but not limited to, liquid solutions or suspensions, tablets, pills, polymeric microcapsules or microvesicles, liposomes, and injectable or infusible solutions. The preferred form depends upon the mode of administration and the particular disease targeted. The compositions also preferably include pharmaceutically acceptable vehicles, carriers, or adjuvants, well known in the art.

Acceptable formulation components for pharmaceutical preparations are nontoxic to recipients at the dosages and concentrations employed. In addition to the anti-cancer agents that are provided, compositions may contain components for modifying, maintaining, or preserving, for example, the pH, osmolarity, viscosity, clarity, color, isotonicity, odor, sterility, stability, rate of dissolution or release, adsorption, or penetration of the composition. Suitable materials for formulating pharmaceutical compositions include, but are not limited to, amino acids (such as glycine, glutamine, asparagine, arginine or lysine); antimicrobials; antioxidants (such as ascorbic acid, sodium sulfite or sodium hydrogen-sulfite); buffers (such as acetate, borate, bicarbonate, Tris-HCl, citrates, phosphates or other organic acids); bulking agents (such as mannitol or glycine); chelating agents (such as ethylenediamine tetraacetic acid (EDTA)); complexing agents (such as caffeine, polyvinylpyrrolidone, beta-cyclodextrin or hydroxypropyl-beta-cyclodextrin); fillers; monosaccharides; disaccharides; and other carbohydrates (such as glucose, mannose or dextrins); proteins (such as serum albumin, gelatin or immunoglobulins); coloring, flavoring and diluting agents; emulsifying agents; hydrophilic polymers (such as polyvinylpyrrolidone); low molecular weight polypeptides; salt-forming counter ions (such as sodium); preservatives (such as benzalkonium chloride, benzoic acid, salicylic acid, thimerosal, phenethyl alcohol, methylparaben, propylparaben, chlorhexidine, sorbic acid or hydrogen peroxide); solvents (such as glycerin, propylene glycol or polyethylene glycol); sugar alcohols (such as mannitol or sorbitol); suspending agents; surfactants or wetting agents (such as pluronics, PEG, sorbitan esters, polysorbates such as polysorbate 20, polysorbate 80, triton, tromethamine, lecithin, cholesterol, tyloxapal); stability enhancing agents (such as sucrose or sorbitol); tonicity enhancing agents (such as alkali metal halides, preferably sodium or potassium chloride, mannitol sorbitol); delivery vehicles; diluents; excipients and/or pharmaceutical adjuvants. (see Remington's Pharmaceutical Sciences, 18 th Ed., (A. R. Gennaro, ed.), 1990, Mack Publishing Company), hereby incorporated by reference.

The above compositions can be administered using conventional modes of delivery including, but not limited to, intravenous, intraperitoneal, oral, subcutaneous, intraarterial, intramuscular, intrapleural, and intrathecal administration. In certain aspects administration can be by perfusion through a regional catheter. Local administration to a tumor is also contemplated.

When administering the compositions by injection, the administration may be by continuous infusion or by single or multiple boluses. For parenteral administration, the anti-cancer agents may be administered in a pyrogen-free, parenterally acceptable aqueous solution.

Once the pharmaceutical composition of the invention has been formulated, it may be stored in sterile vials as a solution, suspension, gel, emulsion, solid, or as a dehydrated or lyophilized powder. Such formulations may be stored either in a ready-to-use form or in a form (e.g., lyophilized) that is reconstituted prior to administration.

If desired, stabilizers can be used, such as sucrose, trehalose, or glycine. Typically, such stabilizers will be added in minor amounts ranging from, for example, about 0.1% to about 0.5% (w/v). Surfactant stabilizers, such as TWEEN®-20 or TWEEN®-80 (ICI Americas, Inc., Bridgewater, N.J., USA), may also be added in conventional amounts.

The components used to formulate the pharmaceutical compositions are preferably of high purity and are substantially free of potentially harmful contaminants (e.g., at least National Food (NF) grade, generally at least analytical grade, and more typically at least pharmaceutical grade). Moreover, compositions intended for in vivo use are usually sterile. To the extent that a given compound must be synthesized prior to use, the resulting product is typically substantially free of any potentially toxic agents. Compositions for parental administration are also sterile, substantially isotonic and made under GMP conditions.

For the compounds of the present invention, alone or as part of a pharmaceutical composition, such doses are between about 0.001 mg/kg and 1 mg/kg body weight, preferably between about 1 and 100 μg/kg body weight, most preferably between 1 and 10 μg/kg body weight.

Therapeutically effective doses will be easily determined by one of skill in the art and will depend on the severity and course of the disease, the patient's health and response to treatment, the patient's age, weight, height, sex, previous medical history and the judgment of the treating physician.

In some methods of the invention the cancer cell is in a patient. The patient may or may not have a solid tumor. In cases where the patient has a solid tumor, embodiments may further involve performing surgery on the patient, such as by resecting all or part of the tumor. Compositions may be administered to the patient before, after, or at the same time as surgery. In additional embodiments, patients may also be administered directly, endoscopically, intratracheally, intratumorally, intravenously, intralesionally, intramuscularly, intraperitoneally, regionally, percutaneously, topically, intrarterially, intravesically, or subcutaneously. Therapeutic compositions may be administered 1, 2, 3, 4, 5, 6, 7, 8, 9, 10, 11, 12, 13, 14, 15, 16, 17, 18, 19, 20 or more times, and they may be administered every 1, 2, 3, 4, 5, 6, 7, 8, 9, 10, 11, 12, 13, 14, 15, 16, 17, 18, 19, 20, 21, 22, 23, 24 hours, or 1, 2, 3, 4, 5, 6, 7 days, or 1, 2, 3, 4, 5 weeks, or 1, 2, 3, 4, 5, 6, 7, 8, 9, 10, 11, 12 months.

Methods of treating cancer may further include administering to the patient chemotherapy or radiotherapy, which may be administered more than one time. Chemotherapy includes, but is not limited to, cisplatin (CDDP), carboplatin, procarbazine, mechlorethamine, cyclophosphamide, camptothecin, ifosfamide, melphalan, chlorambucil, bisulfan, nitrosurea, dactinomycin, daunorubicin, doxorubicin, bleomycin, plicomycin, mitomycin, etoposide (VP16), tamoxifen, taxotere, taxol, transplatinum, 5-fluorouracil, vincristin, vinblastin, methotrexate, gemcitabine, oxaliplatin, irinotecan, topotecan, or any analog or derivative variant thereof. Radiation therapy includes, but is not limited to, X-ray irradiation, UV-irradiation, γ-irradiation, electron-beam radiation, or microwaves. Moreover, a cell or a patient may be administered a microtubule stabilizing agent, including, but not limited to, taxane, as part of methods of the invention. It is specifically contemplated that any of the compounds or derivatives or analogs, can be used with these combination therapies.

In some embodiments, the cancer that is administered the composition(s) described herein may be a bladder, blood, bone, bone marrow, brain, breast, colorectal, esophagus, gastrointestine, head, kidney, liver, lung, nasopharynx, neck, ovary, pancreas, prostate, skin, stomach, testicular, tongue, or uterus cell. In certain aspects the cancer is blood (e.g., lymphoma, leukemia, etc.) or breast cancer.

1. An antibody or antibody fragment that specifically binds a conformational epitope of CTLA-4, PD1, or BTLA and reduces the activity of CTLA-4, PD1, or BTLA, respectively.

2. The antibody of claim 1, wherein the antibody is monoclonal antibody.

3. An anti-CLTA-4 antibody that specifically binds a peptide having an amino acid sequence of SEQ ID NO:2, SEQ ID NO:3, SEQ ID NO:4, SEQ ID NO:5, SEQ ID NO:6, SEQ ID NO:7, or SEQ ID NO:8.

4. The antibody of claim 3, wherein the antibody is monoclonal antibody.

5. An anti-PD1 antibody that specifically binds a peptide having an amino acid sequence of SEQ ID NO:10, SEQ ID NO:11, SEQ ID NO:12, or SEQ ID NO:13.

6. The antibody of claim 5, wherein the antibody is monoclonal antibody.

7. An anti-BTLA antibody that specifically binds a peptide having an amino acid sequence of SEQ ID NO:15, SEQ ID NO:16, or SEQ ID NO:17.

8. The antibody of claim 7, wherein the antibody is monoclonal antibody.

9. A method of treating cancer comprising administering an antibody of claims 1-8.

10. The method of claim 9, wherein the cancer is a blood or breast cancer.

11. The method of claim 9, wherein the cancer is lymphoma.
